(19) United States
(12) Patent Application Publication
(10) Pub. No.: US 2022/0008350 A1
(43) Pub. Date: Jan. 13, 2022

(54) PROCESS FOR ENGINEERING TARGETED NANOPARTICLES

(71) Applicant: University of North Texas Health Science Center, Fort Worth, TX (US)

(72) Inventors: Jamboor K. VISHWANATHA, Fort Worth, TX (US); Andrew GDOWSKI, Fort Worth, TX (US); Amalendu Prakash RANJAN, Fort Worth, TX (US); Anindita MUKERJEE, Fort Worth, TX (US)

(73) Assignee: University of North Texas Health Science Center, Fort Worth, TX (US)

(21) Appl. No.: 17/309,030

(22) PCT Filed: Oct. 16, 2019
(86) PCT No.: PCT/US19/56465

§ 371 Date: Apr. 15, 2021

Related U.S. Application Data
(60) Provisional application No. 62/746,239, filed on Oct. 16, 2018.

(51) Int. Cl. A61K 9/51

(52) U.S. Cl. CPC A61K 9/5176; A61K 9/5192; A61K 9/5153

(57) ABSTRACT

Certain embodiments are directed to methods for making programmable bioinspired nanoparticles (P-BiNP). Nanoparticles are coated with a cell membrane derived from a cell stimulated to express or overexpress a protein identified as being expressed in a target cell, forming a homotypic and organ targeted nanoparticle delivery vehicle.

### CROSS REFERENCE TO RELATED APPLICATIONS

This Application claims priority to U.S. Provisional Patent Application Ser. No. 62/746,239 filed Oct. 16, 2018, which is incorporated herein by reference in its entirety.

### STATEMENT REGARDING FEDERALLY FUNDED RESEARCH

This invention was made with government support under CA194295 awarded by the National Institutes of Health. The government has certain rights in the invention.

### BACKGROUND OF THE INVENTION

### A. Field of the Invention

The invention generally concerns the fields of medicine and drug delivery. In particular, embodiments are directed to cell derived drug delivery systems designed to target drugs to an organ, tissue, or cell of interest.

### B. Description of Related Art

Current systemically administered chemotherapeutics drugs are plagued with multiple inadequacies: (1) Indiscriminate drug delivery of cytotoxic agents can cause severe off target side effects which plays a prominent factor dosing regimens. (2) Many drugs in development suffer from a short half-life, being cleared from systemic circulation rapidly. (3) The combination of the first two factors results in low therapeutic index and sets up drugs for failure. (4) Development of targeted therapy is a major challenge due to complexity and abundance of potential targets, attachment of drugs to targeted agents often affects functionality, and most targeted agents are limited to targeting only a single moiety.

Membrane purifications have been used to coat nanoparticles or as liposomal formulations. But these earlier methods do not utilize bioinformatics to identify clinically relevant targeting molecules and use of this information to alter surface molecules on cells to express the correct signature of molecules. Methods of the current invention are particularly useful in multi-level targeting using cell membranes (e.g., homotypic and organ specific targeting).

There is a need for additional drug targeting compositions and methods.

### SUMMARY OF THE INVENTION

The identification and definition of targeting components of the current invention provide a solution to the current problems associated with limitations in knowledge or number of targeting components for drug delivery. By way of example, the inventors have discovered a process to identify potential targeting components, which results in a delivery vehicle having appropriate targeting properties that enhance delivery of a drug or other therapeutic component to an organ, tissue, or cell. Without wishing to be bound by theory, it is believed that the use of targeting components identified or defined using the current methods results in an improved cell based drug delivery system.

The inventors have created a platform for identifying potential targets based on differences in RNAseq data obtained from different organ sites. Further, the inventors have developed a cell based drug delivery system that utilizes this bioinformatics data to target drugs to the organ of interest. The benefits of this system include: (1) A decrease in off target toxicities by delivery of drug to the site of interest. (2) Due to the cell based coating of the nanoparticle, it will improve circulation time and significantly increase the half-life of drugs being cleared. (3) This platform allows for manipulation of a multi-targeted biomimetic delivery system that also improves drug circulation. (4) The bioinformatics approach gives a greater depth of information so identification of many targets can by identified at once.

In certain aspects the methods of making nanoparticles/liposomes described herein involves identification of proteins/molecules upregulated on the surface of cells that are identified through sequencing or proteomic experimentation or databases. This clinical information is used to guide the selection of stimulation of or alterations to cells prior to isolating the membranes so that the correct signature of molecules is present for biomimetic targeting. In certain aspects the source cell is a cancer or autologous cells that have or have not been manipulated or subjected to stimulation (24,25). This allows the nanoparticles to target specific areas and alter biodistribution. The stimulation or alteration method can target the nanoparticles to specific cancers, to areas of inflammation, to specific organs, etc. Additionally, nanoparticles/liposomes may be made by this method to encapsulate drugs and used for improved delivery of therapeutics.

Certain embodiments are directed to methods for making programmable bioinspired nanoparticles (P-BiNP). Nanoparticles are coated with a cell membrane (programmed membrane) derived from a cell treated in such a manner as to express or overexpress a protein(s) identified as being expressed in a target cell. In certain aspects the cell is treated so that a cell surface profile comprising 1, 2, 3, 4, 5, 6, 7, 8, 9, 10 or more cell surface proteins are expressed or overexpressed. The method can include one or more of the following steps: (a) identifying gene(s) selectively expressed or overexpressed in a tissue or cell to be targeted; (b) isolating a target cell population and stimulating the target cell population under conditions that increase the expression of one or more identified genes forming a stimulated cell population (a cell population with a protein expression pattern such that the cell membranes can be used to target an associated liposome or nanoparticle); (c) isolating the membranes from the stimulated cell population forming stimulated cell membranes; (d) coating polymeric nanoparticles with stimulated cell membranes forming a programmed bioinspired nanoparticle. The gene(s) can be identified by using bioinformatics to evaluate RNA expression in various tissues or cells to be targeted. In certain aspects the identified gene product(s) are selected for homotypic and organ targeting properties. In certain aspects the gene(s) are identified using bioinformatic analysis of RNAseq data from tissues or cell populations. In a particular aspect the gene(s) identified are selectively expressed or overexpressed in a cancer metastasis, e.g., a bone, liver, brain, or lymph node metastasis of any type of cancer. In particular examples the gene(s) identified can include, but is not limited to integrin αVβ₃ or various other integrins or cell surface or extracellular proteins.

An isolated target cell population can be treated or stimulated to express or overexpress the identified gene(s), in particular aspects the stimulation is performed in vitro. Stimulating an isolated target cell population can results in a 2, 4, 6, 8, 10 fold or more increase in expression of a gene or genes identified as being selectively expressed or overexpressed in a tissue or cell to be targeted. In certain aspects stimulating conditions increase expression of at least integrins such as integrin αVβ₃ in the stimulated cell population.

Isolated target cell population stimulation can be performed by using various reagents and/or conditions known in the art to stimulate the expression of 1, 2, 3, 4, 5, 6, 7, 8, or more genes in a target cell population. For example a targeted cell population can be contacted with a protein ligand that binds receptors on the cell surface which in turn activate various signaling pathways in the target cell leading to a programmed expression of proteins on the cell surface. The term programmed as used herein refers to exposing a cell to defined conditions in order to influence expression of proteins.

In certain aspects the tissue or cell to be targeted includes or is a cancer cell, e.g., a prostate cancer cell or a metastasis thereof.

In certain aspects the nanoparticle can be coated by co-extrusion of stimulated cell membranes and nanoparticles, forming a P-BiNP. The P-BiNP can be coated at ratios of 0.25:1, 0.5:1 to 1:1 weight of cell membrane protein to weight of nanoparticle, including all values and ranges there between. In particular aspects nanoparticles are PLGA nanoparticles.

Certain embodiments are directed to methods of treating a subject comprising administering to the subject P-BiNP as described herein.

Other embodiments are directed to P-BiNP produced using the method described herein.

The term “nanoparticle” refers an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the therapeutic nanoparticles that contain or are coupled to a therapeutic agent. In certain aspects the therapeutic agent is an anticancer agent. In certain aspects the anticancer agent is a chemotherapeutic agent.

The term “chemotherapeutic agent” refers a molecule that can be used to reduce the rate of cancer cell growth or to induce or mediate the death (e.g., necrosis or apoptosis) of cancer cells in a subject (e.g., a human). In non-limiting examples, a chemotherapeutic agent can be a small molecule, a protein (e.g., an antibody, an antigen-binding fragment of an antibody, or a derivative or conjugate thereof), a nucleic acid, or any combination thereof. Non-limiting examples of chemotherapeutic agents include: cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, axathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

“RNAseq” refers to RNA Sequencing, or more specifically, total transcriptome sequencing, i.e., the sequencing of all messenger RNA in a sample.

The term “transcriptome” refers to the set of all RNA molecules, including mRNA, rRNA, tRNA, and other non-coding RNA produced in a cell or in a population of cells.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and/or claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates the concept for personalized nanoparticles engineered with enhanced ability for synchronized selective organ localization and homotypic binding for with potential clinical relevance.

FIG. 2. Membrane Fraction Purification. (Left) Western blot of LNCap and C4-2B cell lines were used to verify that purified membrane did not contain nuclear or mitochondrial components that the whole cell lystate (WCL) contained. (Right) coomassie blue stain was used to verify to purified membrane of both C4-2B and LNCaP cell lines retained panel of membrane proteins.

FIG. 3. Nanoparticle coating and stability study. PLGA nanoparticles were coated with different ratios of cell membrane to nanoparticle polymer to determine the optimal amount of cell membrane coating needed to make the nanoparticle stable. Coated nanoparticles were placed in normal saline solution for various time point. An increase in size is indicative of aggregation and insufficient coating of nanoparticles to remain stable in ionic solution. Mean±SEM. (Inset) shows size range from 0-400 nm.

FIG. 4. Membrane Orientation. To determine whether extracellular side of membrane was orienting correctly on nanoparticle surface, an immuno-nanoparticle assay was performed. Nanoparticles were tagged with nile red, incubated with either antibody against intracellular epitope of EGFR (top row) or antibody against extracellular epitope of EGFR, washed, and then incubated with protein A/G agarose beads. Nanoparticles treated with antibody against extracellular epitope of EGFR showed binding affinity to protein A/G agarose beads indicating that membrane coating the nanoparticles is in correct orientation.

FIG. 5. Fluorescent microscopy of CCNP cellular uptake. Either bare nanoparticles labeled with nile red (top) or cancer membrane coated also labeled with nile red were incubated with C4-2B cells. Additionally, during the preparation process, the CCNPs were tagged with Pkh26 dye to label the lipid content of the cell membrane coating the nanoparticles. There was increase uptake in the C4-2B cell line with the CCNP nanoparticles compared to the non-labeled nanoparticles.

FIG. 6. Flow Cytometry Cellular Uptake Study. (Top) Representative examples of nanoparticle uptake experiment. Nanoparticles were tagged with nile red dye and bare-NP or CCNP were used to treat C4-2B cells for 1 hour. Media was washed and cells were lifted from dish and processed through flow cytometry and gated to detect nile red flourescence. (Bottom) quantification of triplicate experiments run showing increased uptake of CCNPs in C4-2B cells compared to bare-NP (PLGA). (n=3). Mean±SEM. * P<0.05.

FIG. 7. MTT Cell viability assay. CCNPs that were either stimulated or non-stimulated with CXCL12 were used to treat C4-2B cells for 72 hours. Stimulated CCNPs showed decreased cell viability than the non-stimulated CCNPs. (n=3). Mean±SEM.

FIG. 8. Illustrates a clinical scenario of programmable bioinspired nanoparticles. (1) Cells are isolated from patient's biopsy. (2) Cells are grown in a petri dish and programmed by (3) stimulation with CXCL12 to enhance homotypic binding and bone adhesion ability. (4) Membrane is isolated from programmed cancer cells and used to coat (5) nanoparticles with drug or imaging agent cargo. (6) Programmable bioinspired nanoparticles (P-BiNPs) are injected back into patient with enhanced bone homing and homotypic binding.

FIGS. 9A-E. αVβ₃ identified as target for enhanced homotypic binding and bone adhesion. (A) Age of patients at diagnosis in database. Total patients involved in study (n=150). Tumor samples with mRNA analyzed (n=118). (B) Percentage distribution of prostate cancer metastatic locations, the top three metastatic sites were bone, liver and lymph node. (C) Heat map of mRNA z-score organized by metastatic location vs. genes identified by gene set enrichment analysis that are involved in homotypic cell-cell adhesion. (D) Increased expression of the beta 3 (ITGB3) subunit of αVβ₃ integrin in bone metastatic prostate cancer compared to other metastatic sites. (E) Quantification of ITGB3 expression levels (RNA Seq RPKM) of the three most common metastatic locations. Bone has a significantly higher level of expression of ITGB3 compared to liver and lymph nodes. Mean±SEM. **** P<0.0001.

FIGS. 10A-B. Programming cancer cells to have higher surface expression of αVβ₃. (A) Representative immunocytochemistry images of C4-2B cells at various time points after stimulation. (Top) Dapi. (Middle) αVβ₃ expression. (Bottom) Overlay. (B) Quantification of average fluorescent intensity per cell with increased αVβ₃ surface expression after stimulation (n=3). * P<0.05. Mean±SEM.

FIGS. 11A-D. Nanoparticle characterization. (A) Size and PDI of nanoparticles as measured by dynamic light scattering. There was a clear increase in size when comparing bare nanoparticles to nanoparticles coated with membranes. (B) Zeta potential measurements resulted in a less negative zeta potential when comparing bare nanoparticles to membrane coated nanoparticles. (C) Transmission electron micrograph (TEM) of BiNP nanoparticles showing coating with cancer cell membrane. (D) Stability study performed for 7 days demonstrates the nanoparticles are not aggregating in solution. Samples run in triplicate. Mean±SEM. ** P<0.01, *** P<0.0005, **** P<0.0001.

FIGS. 12A-C. Programmed bioinspired nanoparticles have increased uptake into cancer cells. (A) Representative images of flow uptake experiment. Nanoparticles were tagged with nile red dye and incubated with C4-2B prostate cancer cells or human fibroblast cells for 1 hour and uptake was assessed through flow cytometry gated to detect nile red fluorescence. (B) Quantification of triplicate experiments showing highest uptake in C4-2B cells when incubated with P-BiNPs. (Inset) Nanoparticle uptake in fibroblast cells. (C) MTT cell viability assay after treatment with increasing concentrations (0.2-20.0 mg/ml) of BiNP or P-BiNP resulting in decreased cell viability of P-BiNPs at equivalent treatment dosage as BiNPs after 72 hours. (n=4). Mean±SEM. * P<0.05, ** P<0.01, **** P<0.0001.

FIGS. 13A-E. Enhancement of bone homing via programmable bioinspired nanoparticles. (A) Relative percentage difference of organ localization between P-BiNP vs. BiNP after tail vein injection showing highest P-BiNP localization in heart and bone. (B) Representative image overlay showing difference in nanoparticle organ localization. A relative reduction in P-BiNP NIR signal in the organ is indicated in red or increase in P-BiNP in the organ is represented by green compared to BiNP. (C) Absolute fluorescent values for the two organs (heart and bone) with increased P-BiNP signal after injection demonstrating higher levels of P-BiNP in the bone compared to heart. (D) High resolution scan of hind limbs for sensitive detection and localization of either dye, BiNP, or P-BiNP. Green=800 nm wavelength emission of NIR dye. (E) Quantification of high resolution scans showing highest signal in P-BiNP group. (n=4). Mean±SEM. * P<0.05, ** P<0.001.

FIG. 14. Table of homotypic cell-cell adhesion gene expression in prostate cancer patients' tumors. RNAseq data set mined from www.cbioportal.org with fold difference in expression between bone and liver as well as bone and lymph node in metastatic prostate cancer patients. Table arranged by fold difference levels with dark panel having highest fold increase in bone metastatic lesions. ITGB3 is in red and was identified and selected based on high relative fold increased levels in bone metastatic prostate tumors, membrane surface expression, and known role in bone metastasis. * P<0.05, ** P<0.01, *** P<0.0005, **** P<0.0001.

FIG. 15. Ratio of cell membrane to nanoparticle polymer. PLGA nanoparticles were coated with different ratios of cell membrane to nanoparticle polymer to determine optimal amount of cell membrane coating needed to stabilize nanoparticles as measured through stability of size. Nanoparticles were placed in PBS solution and size was measured after 12 hours. An increase in size is indicative of aggregation. (WCL=whole cell lysate). (n=3). Mean±SEM.

FIG. 16. Co-culture spheroid penetration assay. C4-2B (prostate cancer cells) and HFF-1 (fibroblasts) were co-cultured in non-adherent dish. After 3D spheroid formation, incubation was performed with either BareNP (no membrane coating) or P-BiNP, both labeled with nile red fluorescent dye. Deconvoluted microscopy showed P-BiNP were able to thoroughly penetrate spheroids after 3 hours of incubation. Blue=Dapi. Red=nile red in core of NPs.

FIG. 17. Cytotoxic effect of PBiNPs with the highest dose on normal prostate epithelial cells PWR1E. Data represented as mean±SE.

FIG. 18A-18B. SDF-1 stimulation of human bone metastatic prostate cancer cells and avb3 integrin. C42B cells were incubated with SDF-1 (200 ng/ml) for indicated time intervals, and the proteins levels of αv, β3, phosphor β3 integrin was determined using western blot. (A) Western blots. (B) Graphical representation of Western blot results.

### DETAILED DESCRIPTION OF THE INVENTION

Targeting therapeutic agents to specific organs in the body remains a challenge despite advances in the science of systemic drug delivery. To demonstrate the methods described herein and the resulting composition the inventors have engineered a programmable bioinspired nanoparticle (P-BiNP) delivery system to simultaneously target the bone for example and achieve self-recognition of homotypic tumor cells by coating polymeric nanoparticles with programmed cancer cell membranes. This bioinspired approach incorporates relevant clinical bioinformatics gene expression data to guide the design and enhancement of biological processes that these nanoparticles are engineered to mimic. To achieve this, an analysis of RNA expression from therapeutically target cell such as a metastatic prostate cancer can identify gene(s), e.g., ITGB3 (a subunit of integrin αVβ₃) in metastatic prostate cancer cells, as highly overexpressed in patients, e.g., with bone metastasis. In this particular case cancer cells were stimulated to increase this integrin expression on the cell surface and these membranes can be used to coat cargo carrying polymeric nanoparticles. Physicochemical optimization and characterization of the P-BiNPs showed desirable qualities regarding size, zeta potential, and stability. In vitro testing confirmed enhanced homotypic binding and uptake in cancer cells. P-BiNPs also demonstrated improved bone homing and retention in vivo with a murine model. This approach of identifying clinically relevant targets for dual homotypic and organ targeting has tremendous potential as a strategy for treatment and imaging modalities in diseases that affect the bone as well as broader implications for delivering nanoparticles to organs, tissues, and/or cells of interest.

Nanoparticles have the potential to improve drug delivery through targeting either by passive or active means. Passive targeting utilizes the small size of nanoparticles to achieve enhanced permeability and retention (EPR) in tumors through penetration of leaky vasculature and accumulation in the tumor due to inadequate or absent lymphatic drainage [1]. In comparison, active targeting requires the use of a ligand against an entity that is overexpressed on cancer cells to allow increased binding and uptake of the nanoparticle, thereby improving cargo uptake into the cancer cell [2]. In the preclinical setting, both passive and active strategies have been employed effectively to enrich nanoparticle concentration in tumors. However, the active targeting approach for nanoparticles has been generally less successful, especially in the clinical setting.

In some embodiments, therapeutic nanoparticles may or may not contain a core of a magnetic material (e.g., a therapeutic magnetic nanoparticle). In some embodiments, the therapeutic nanoparticles described herein do not contain a magnetic material. In some embodiments, a therapeutic nanoparticle can contain, in part, a core containing a polymer (e.g., poly(lactic-co-glycolic acid)). Skilled practitioners will appreciated that any number of art known materials can be used to prepare nanoparticles, including, but are not limited to, gums (e.g., Acacia, Guar), chitosan, gelatin, sodium alginate, and albumin. Additional polymers that can be used to generate the therapeutic nanoparticles described herein are known in the art. For example, polymers that can be used to generate the therapeutic nanoparticles include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate and polycaprolactone.

In some embodiments, the magnetic material or particle can contain a diamagnetic, paramagnetic, superparamagnetic, or ferromagnetic material that is responsive to a magnetic field. Non-limiting examples of therapeutic magnetic nanoparticles contain a core of a magnetic material containing a metal oxide selected from the group of: magnetite; ferrites (e.g., ferrites of manganese, cobalt, and nickel); Fe(II) oxides, and hematite, and metal alloys thereof. The core of magnetic material can be formed by converting metal salts to metal oxides using methods known in the art (e.g., Kieslich et al., Inorg. Chem. 2011). In some embodiments, the nanoparticles contain cyclodextrin gold or quantum dots. Additional magnetic materials and methods of making magnetic materials are known in the art. In some embodiments of the methods described herein, the position or localization of therapeutic magnetic nanoparticles can be imaged in a subject (e.g., imaged in a subject following the administration of one or more doses of a therapeutic magnetic nanoparticle).

This difference between preclinical success and clinical translation may be due to a variety of factors. First, many ligand coating strategies for targeted nanoparticles involve difficult and complicated chemical conjugation strategies which can alter the ligand's affinity for its target [3]. Second, often the choice of target in the preclinical setting is made without the input of clinically relevant targets and data from patients. Third, an often-neglected consideration in engineering targeted nanoparticles is the impact of ligand surface density on binding as well as uptake into cells [4]. Fourth, the heterogeneity of surface markers on cancer cells often diminishes the ability to efficiently target all cells which make up the tumor [5].

Considering these factors, the inventors have designed and engineered a biologically inspired strategy to simultaneously deliver nanoparticles to the bone with increased targeted cell uptake. The primary goal of this approach was inspired by prostate cancer cells' ability to home to the bone during the metastatic process. The progression of bone metastasis is quite complex and involves multiple coordinated events including escape from the primary tumor, survival in systemic circulation, and the ability to home to the bone microenvironment [3,6]. This nanoparticle delivery system seeks to mimic the latter two processes so that nanoparticle cargo can be transported and retained in the bone. Coating nanoparticles with biological membranes has been shown to increase the circulation time of the nanoparticles due to the improved biocompatibility in systemic circulation [7]. In addition, specific factors involved in the homing process that are present on the membranes can be enhanced through ex vivo biological methods and thus eliminate the need for traditional challenging chemical conjugation schemes. This alternative strategy allows fusing the cell membranes to core nanoparticles as a simple method to create a complex biocompatible system for improved targeting ability.

Several factors involved in cancer cells homing to the bone have been described in the in vitro setting [8]. However, limited data exists demonstrating in vivo validation of these altered genes and proteins. Thus a combination of in vitro data and patient RNAseq data was evaluated in selecting factors to enhance. A bioinformatics analysis of an RNAseq database from prostate cancer patients with metastasis to various sites was used to establish differentially expressed factors in patients with bone metastasis. Increased mRNA expression was used as an indicator of factors involved in the bone metastatic process. ITGB3 was identified as having increased expression in the tumors of patients with bone metastatic prostate cancer but not metastasis to other common locations such as the liver and lymph nodes. ITGB3 encodes an important subunit of the integrin αVβ₃ which is a critical factor contributing to the ability of prostate cancer cells to specifically home and bind to endothelial in the bone [9]. Increased membrane expression of this integrin occurs when prostate cancer cells are stimulated by the chemokine factor, C-X-C motif chemokine ligand 12, (CXCL12) that originates from osteoblasts in the bone [9]. The inventors contemplate that using this signaling pathway could be used to stimulate or program the BiNPs to have enhanced bone homing and retention ability.

Another objective of the P-BiNPs is to achieve selective targeting to specific cells so that once the nanoparticles have homed to the organ of interest, there will be preferential uptake into the identified cells. This strategy has the potential to improve delivery of therapeutic agents, enhance imaging agents, and decrease off-target side effects.

Bioinformatics Target Identification. In the context of cancer metastasis, a gene set enrichment analysis can be performed to identify genes associated with both biological adhesion and cell surface, resulting in the identification of an overlap in genes. Patient tumor RNAseq raw data can be accessed or obtained for use in identifying genes that have statistically significant expression from those genes identified at common sites of metastasis (bone, liver, lymph node). Groups can be stratified into bone, liver, or lymph node based on highest mean z-score. Ranked importance of gene expression for each metastatic group can be based on multiple comparisons test or other known methods.

In a specific example, gene set enrichment analysis was performed to identify genes associated with both biological adhesion (n=1032) and cell surface (n=757). The results identified overlap in 210 genes. Open source patient tumor RNAseq raw data accessed cbioportal.org with patient and sequencing data from Metastatic Prostate Cancer, SU2C/PCF Dream Team cohort was utilized to identify 69 genes that had statistically significant expression from those genes identified at common sites of metastasis (bone, liver, lymph node.) Groups were stratified into bone, liver, or lymph node based on highest mean z-score. Ranked importance of gene expression for each metastatic group was based on multiple comparisons test. Below are the list of identified genes from most important in disease state to least:

Bone Metastasis: ITGB3, SRPX, PSTPIP1, CD33, CCR1, ITGAM, NTSE, ITGA2B, CD63, OTOA, TGFB1, CD36, EMR1, ITGB2, MICA, C10orf54, TNN, CD58, TPBG, CD4, LILRB2, DSCAML1, SPN, TSPAN32, S1PR1, EPHA2.

Liver Metastasis: SLC7A11, FGB, AMBP, FGG, NRCAM, TNFRSF12A, EMP2, ANXA9, ASTN1, L1CAM, SCARB1, NCAM1, MYH9, ROBO1, CEL, BMP10, ACE2, EPCAM, AIMP1, APP, ITGB8, ROBO2, PKHD1, STAB2, LRFN3, NRXN1, B4GALT1, PVR, ITGA1, RHOB, KIT, ITGB6, CLSTN3, TNFSF18, SRPX2.

Lymph Node Metastasis SELP, MYH10, HSPD1, ITGB5, FZD4, CD34.

As proof of principle ITGB3 was chosen due to its critical role in bone metastasis. Literature was searched for methods of stimulating proteins associated with ITGB3 (bioinformatics pathway analysis could also be used for this step). ITGB3 is a subunit of the protein avB3 and can be stimulated with treatment by SDF-1. Conditions for stimulation with SDF-1 and verification of increased expression of avB3 on cell membrane has been shown.

In other methods quantitative proteomic analysis can be used to identify other proteins with altered expression after stimulation.

A method for isolating a pure membrane cell fraction has been optimized. Membrane coating of nanoparticles has been optimized for maximum stability.

Increased uptake of membrane coated nanoparticles in cancer cells has been demonstrated.

The inventors have demonstrated decreased cell viability when stimulated nanoparticles are used to treat cancer cells. Targeting can be demonstrated using appropriate animal experiments.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Programmable Bioinspired NanoParticles (P-BiNPs)

### A. Results

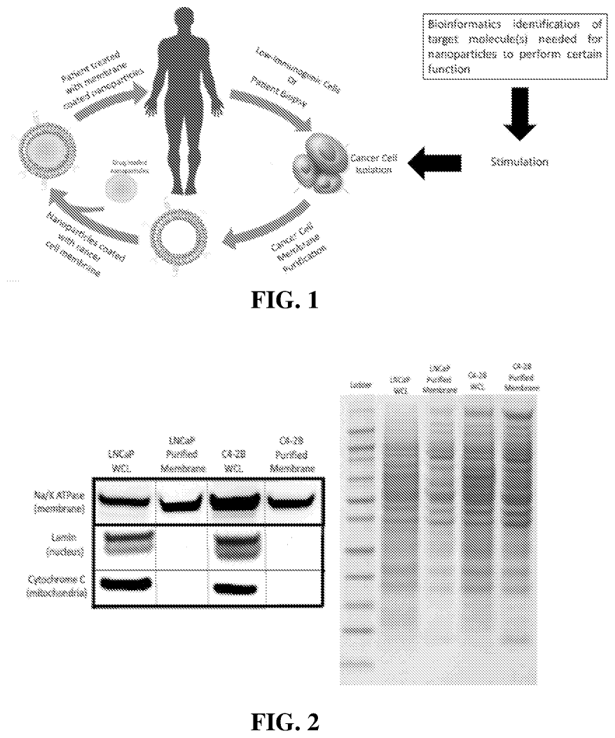

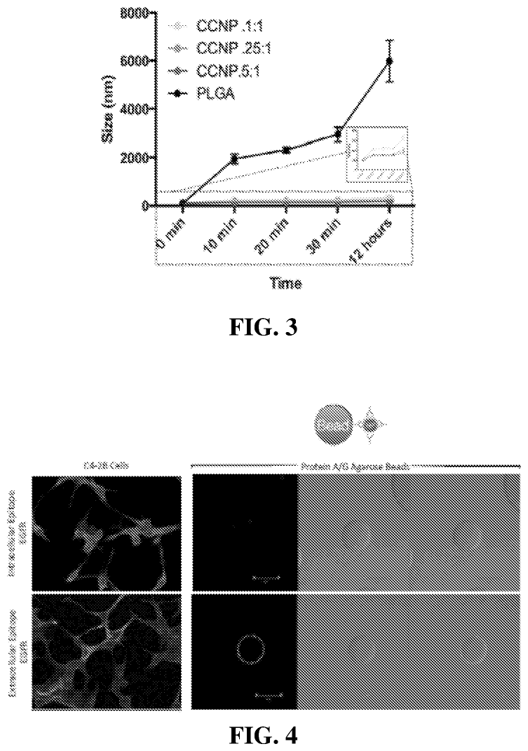

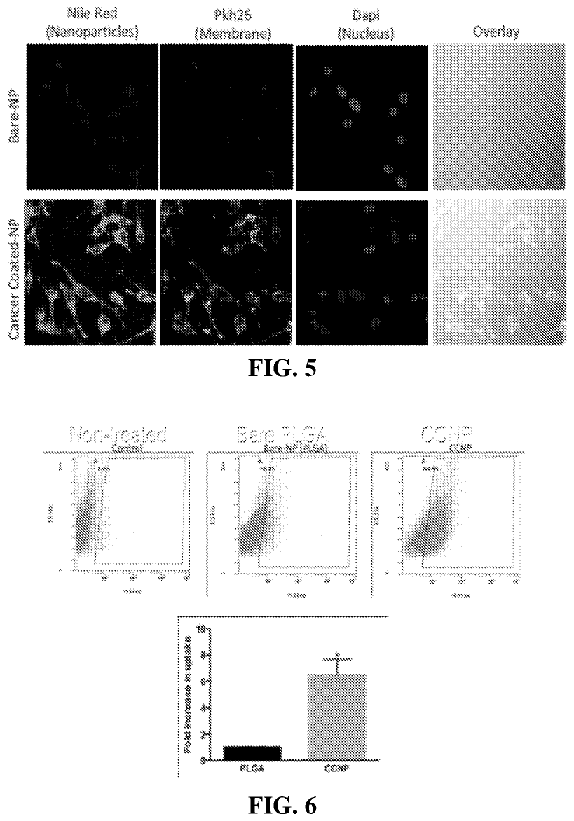

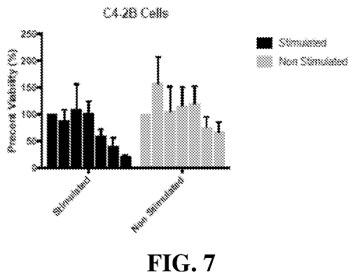

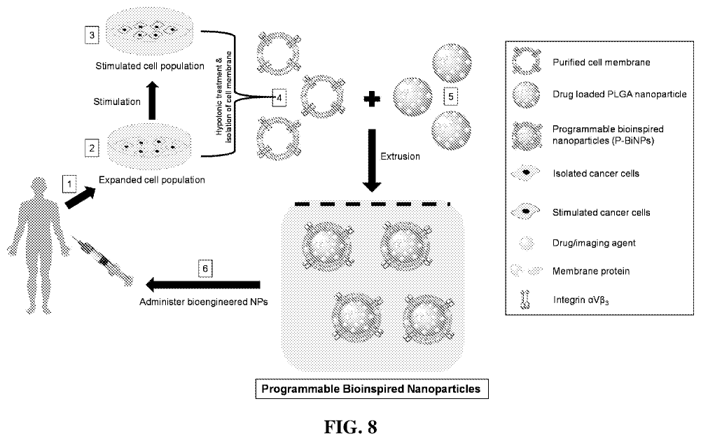

Target Identification and Validation. Several studies have demonstrated the principle of homotypic targeting, through which nanoparticles can be coated with various cancer cell lines and result in higher uptake into homologous tumors [10-13]. However, the factors responsible for the phenomena of homotypic binding have primarily been unexplored especially in the clinical context. Further, it has not been established whether stimulating a factor that is important in homotypic binding can simultaneously be exploited for preferentially targeting nanoparticles to an organ of interest. Thus, one goal of the described method is to provide proof of concept for personalized nanoparticles engineered with enhanced ability for synchronized selective organ localization and homotypic binding for with potential clinical relevance (FIG. 1 and FIG. 8). Prostate cancer was chosen as the prototype for this proof of concept due to its high prevalence, bone homing ability in the metastatic setting, and low immunogenicity.

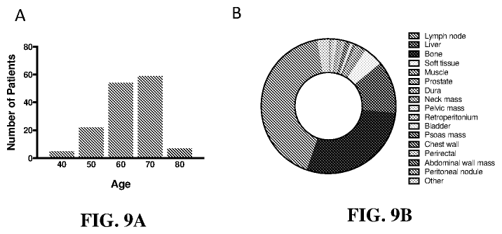

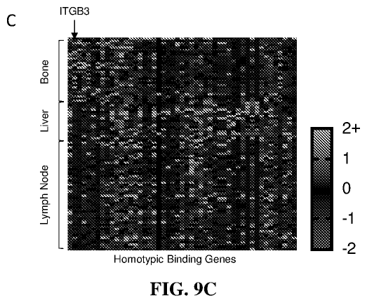

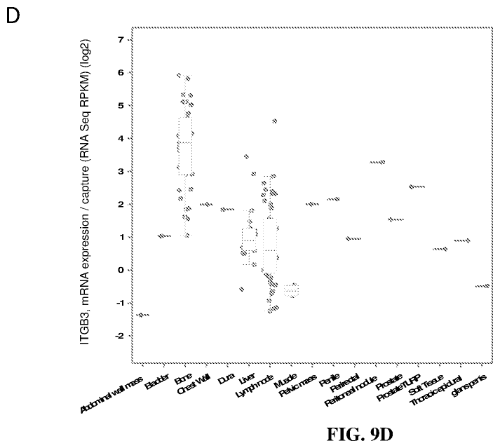

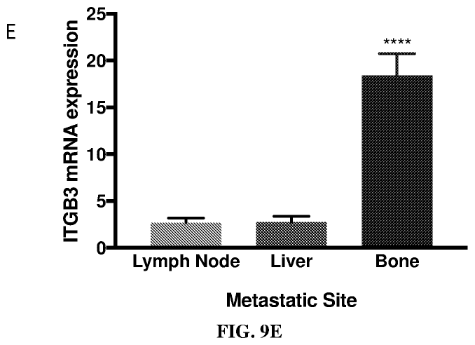

A gene set enrichment analysis (GSEA) search identified 55 genes that were enhanced in the process of homotypic cell-cell adhesion. Subsequent bioinformatics analysis of a RNAseq database from metastatic prostate cancer patient samples (n=118) identified ITGB3 as being significantly increased in bone metastatic lesions compared to metastases from other sites such as liver, lymph nodes, or other organs (P<0.0001) (FIG. 9). ITGB3 is a subunit of integrin αVβ₃ and was selected as a clinically relevant target protein for which enhancement could impact nanoparticle delivery to bone and homotypic binding in the final P-BiNP.

CXCL12 was identified through a literature search as a ligand that can increase surface expression of integrin αVβ₃ through binding to CXCR4 on cancer cells in culture. Further, the increased expression αVβ₃ has been shown to increase binding of prostate cancer cells specifically to bone marrow-derived endothelial cells in experimental models [9]. In this example, the C4-2B prostate cancer cell line was used for proof of concept. This prostate cancer cell line has known ability to create bone lesions in a mouse model [14,15].

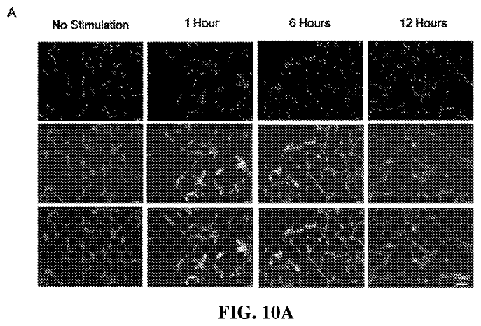

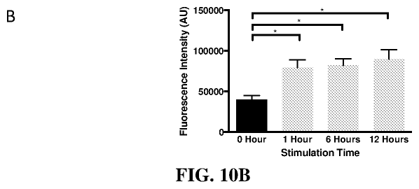

C4-2B prostate cancer cells were first stimulated with CXCL12 for varying amounts of time and the expression level of αVβ₃ was determined after specified time points through immunocytochemistry. There was an approximately 2-fold increase in expression level of αVβ₃ after the first hour of stimulation and this remained constant for the 12-hour duration of the experiment (FIG. 10). Thus, 1 hour was chosen as the length of time needed to program the cells for increased surface expression of αVβ₃ in subsequent experiments.

Preparation and Characterization of Nanoparticles. After cancer cells were programmed to have increased expression of αVβ₃, our next step was to isolate those cancer cell membranes and optimize the coating and physical properties of the nanoparticles. The inventors first isolated the membranes by a differential centrifugation technique to harvest the lipid and embedded protein components. Western blot verified the purity of the membrane fraction and confirmed the presence of Na/K ATPase, a membrane marker, in both the whole cell lysate and purified membrane samples. As expected, nuclear marker (lamin) and mitochondrial marker (cytochrome C) were absent in the purified membrane lysates but present in the whole cell lysates. Additionally, coomassie blue stain displayed a considerable profile of membrane proteins that were expressed after membrane purification process in both C4-2B and LNCaP prostate cancer cell lines. These proteins may also have an impact on the homotypic binding and bone adhesion of the nanoparticles beyond the known functional influence of αVβ₃ integrin.

Next, the ideal amount of cell membrane to coat the nanoparticle was explored using a nanoparticle stability assay. Various ratios of nanoparticle to cell membrane were coated and then introduced into an ionic solution of PBS to induce aggregation of non-coated or partially coated nanoparticles as measured by an increase in hydrodynamic diameter [10,16]. Nanoparticles coated with ratios of 0.25:1 and 0.5:1 (weight of cell membrane protein to weight of polymer) were most stable after being introduced into PBS. Whereas, non-coated PLGA nanoparticles and P-BiNPs with membrane to polymer ratio of 0.1:1 tended to aggregate. Thus, the ratio 0.5:1 was selected for further experiments. For the nanoparticle core, the inventors used Poly(DL-lactide-co-gylcolide) (PLGA) polymeric nanoparticles due to their negative zeta potential, biocompatibility, and high encapsulation efficiency of hydrophobic molecules that can be loaded for therapeutic or imaging purposes [17].

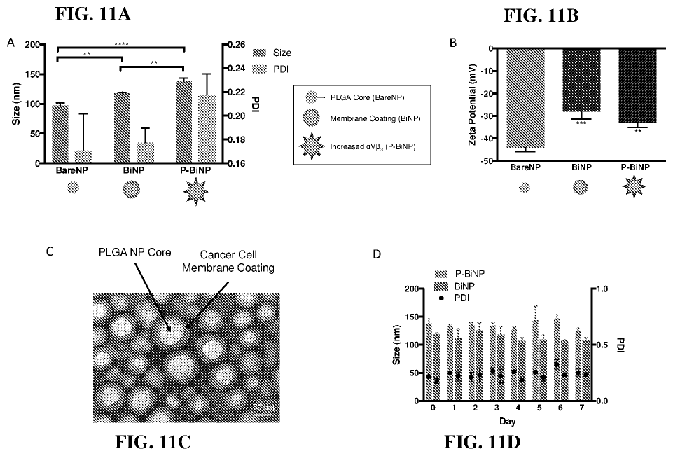

Nanoparticle size, as measured by dynamic light scattering (DLS), showed an expected size increase after being coated with the cancer cell membrane. The initial size of non-coated nanoparticles was 97.2 nm and increased to 117-138 nm when coated (P<0.0001) (FIG. 11). Transmission electron microscopy (TEM) was used to visualize and verify the membrane coating on the NP (FIG. 11). Zeta potential also significantly changed as the nanoparticles were coated with the cancer cell membrane from −44 mV when uncoated, to −28 mV through −33 mV in the coated nanoparticles (FIG. 11). Other cell types were tested and found to have similar trends in both increased size and zeta potential measurements when nanoparticles were coated. Also, stability measurements of both the BiNP and the P-BiNP show similar constancy in both size and PDI over the time course of one week when stored at 4° C.

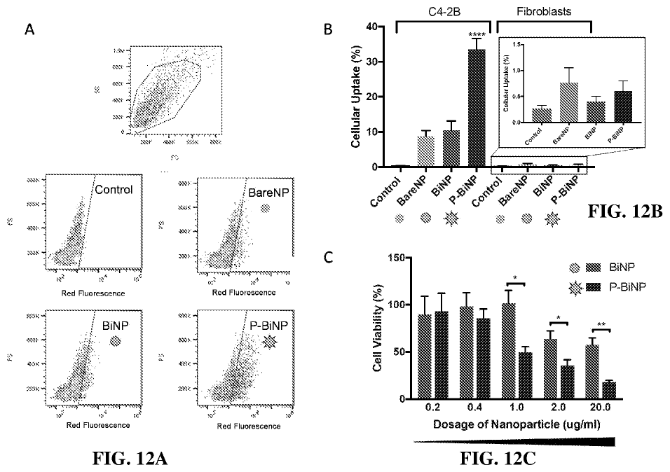

In Vitro Uptake and Cytotoxicity of P-BiNPs. Some cancer cells and nanoparticles coated with cancer cells have been reported to exhibit homotypic targeting properties in which they self-recognize tumor cells of the same type [11,12,18]. To determine whether this self-recognition could be enhanced through programming cells to increase expression of αVβ₃ by a natural stimulation process both C4-2B and fibroblasts were treated with either BareNPs, BiNPs, or P-BiNPs that were derived from C4-2B membranes and fluorescently tagged. Flow cytometry and immunocytochemistry were used to measure the uptake in the cells. P-BiNPs had a much higher uptake in the C4-2B cell line as measured through flow cytometry (FIG. 12). The approximately 4-fold increased uptake with the P-BiNP group compared to the BiNP group indicates that the stimulation process is an important factor for enhancing the nanoparticle cellular uptake.

This increased uptake was also studied by labeling both the membrane component and the nanoparticle core with two separate fluorescent dyes of different peak emission wavelengths before synthesis to determine if both the membrane and nanoparticle core were taken up in cancer cells at the same time. It was found that there was indeed simultaneous uptake in the cells and co-localization of both dyes after confocal microscopy imaging. Moreover, when 3D prostate cancer spheroids were created, the P-BiNP had no issue thoroughly penetrating the spheroid.

Increased cell uptake into cancer cells and tumors should result in improved cytotoxicity of chemotherapy delivered to the cells as the molecules are transported inside the cells more efficiently. The inventors tested whether programming the nanoparticles to have increased αVβ₃ on their surface would translate to having increased cytotoxic effects compared to BiNP with no stimulation using a MTT cell viability assay. The microtubule inhibitor, Cabazitaxel, was encapsulated within the nanoparticles and cells were either treated with BiNPs or P-BiNPs. Cabazitaxel was chosen as a model drug because in addition to being FDA approved for metastatic prostate cancer, it has lower substrate affinity for the ATP-dependent drug efflux pump glycoprotein (P-gp) that is commonly up-regulated in metastatic and chemotherapy-resistant cancers [19]. Thus, Cabazitaxel is less likely to be pumped out of the advanced tumor cells. P-BiNPs showed decreased cell viability compared to BiNP (FIG. 12) hence demonstrating that the natural stimulation process causing higher expression levels of αVβ₃ can improve the efficacy of chemotherapy via improvement of chemotherapy delivery inside the cell.

In Vivo Bone Homing and Adhesion of P-BiNPs. Bolstering homotypic targeting was one of the design goals of this nanoparticle. The second objective was enhancing the ability of the P-BiNP to bind to the bone through a bioinspired and clinically relevant mechanism. This was achieved by identification of αVβ₃ integrin playing an important role in bone homing of prostate cancer. This was evidenced by the inventors' findings of high αVβ₃ expression levels in tumors of prostate cancer patients who had bone metastasis but much lower levels of metastasis to other locations like the liver and lymph nodes. The role of this protein has been studied in vitro in the context of tumor cell adhesion to bone components such as vitronectin, bone sialoprotein, osteopontin, and other bone extracellular matrix factors [20-23].

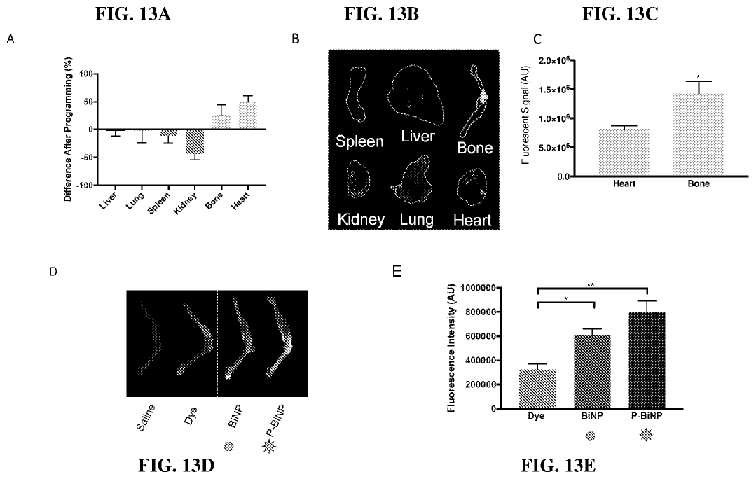

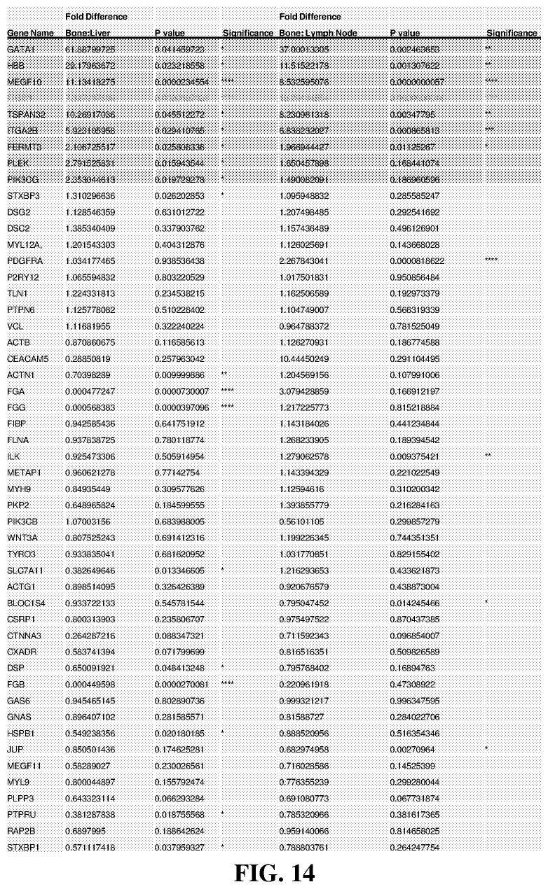

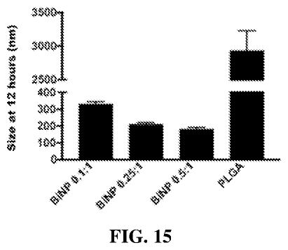

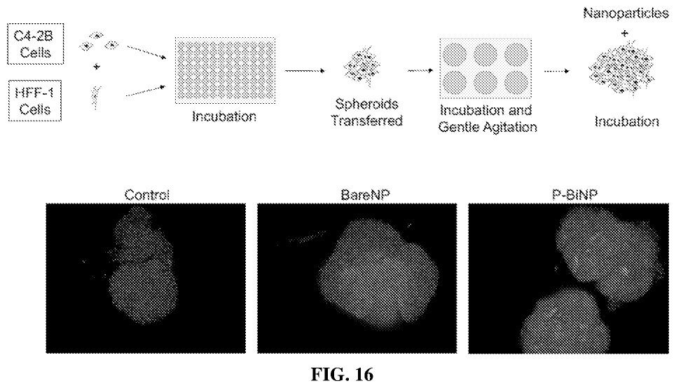

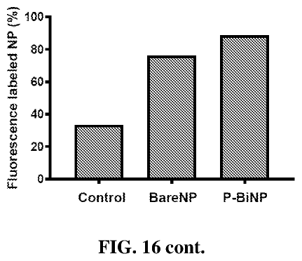

Nanoparticles were injected intravenously via the tail vein in mice. This route of administration tests whether the P-BiNPs mimic the bone homing ability observed in prostate cancer cells. It was found that after allowing the nanoparticles to circulate for two hours, the P-BiNPs groups had a higher fluorescent signal in the bone, indicating an increased ability to home to the bone compared to BiNP and dye groups. As expected, there were also elevated levels of nanoparticles in the liver and lungs (FIG. 13).

Adhesion and nanoparticle retention were tested by utilizing an intraosseous injection technique. This injection method allows for direct assessment of adhesion and retention of the BiNP and the P-BiNPs in the bone. It also removes confounding variables that may influence the nanoparticle behavior when injected systemically. BiNP and P-BiNPs were labeled with a NIR dye and injected directly into the mouse tibia (FIG. 13A (inset)). The P-BiNP demonstrated longer retention and half-life in the bone through live animal imaging up to 72 hours (FIG. 13). This highlights the need for programming and activation of αVβ₃ prior to nanoparticle coating of the membrane as the BiNP and the dye itself were equivalent regarding retention in the bone.

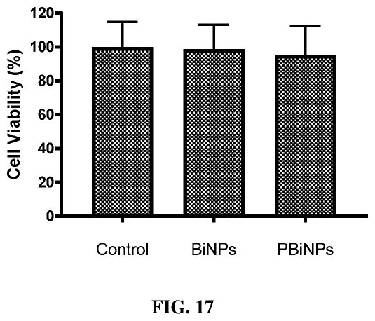

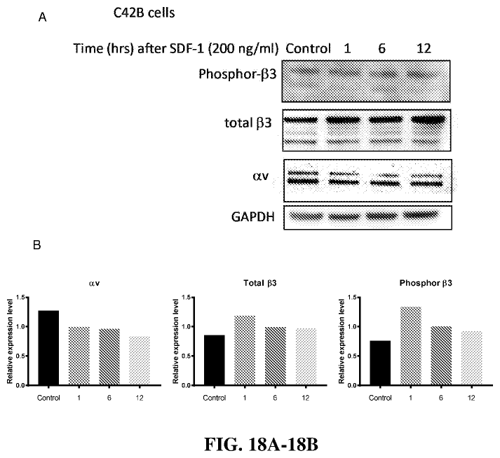

Cytotoxicity of PBiNPs on normal cells. To examine the cytotoxic effect of the PBiNPs against normal prostate epithelial cells (PWR1E), cell toxicity assay was performed. Normal epithelial prostate cells were treated with highest dose of the PBiNPs for 24 hours. No significant cytotoxic effect was observed in the normal epithelial prostate cells at the highest concentration (FIG. 17.

Western blot Analysis for Expression of Proteins. Western blot analysis was conducted for αVβ₃ quantification, 1 hr, 6 hr, and 12 hr. The expression of Integrin αVβ₃ using western blotting is not completely aligned when compared to flow cytometry data. This can be attributed to the fact that phosphor-β3 integrin observations using western blotting included only one site Tyr773 and not all phosphorylation sites.

### B. Materials and Methods

Bioinformatics Data. Gene ontology consortium (URL www.geneontology.org) query identified 55 potential targets involved in homotypic cell-cell adhesion. These targets were cross-referenced with RNAseq expression levels from 118 patients with metastatic prostate cancer to various organ locations. Multiple hits were identified for upregulated mRNA expression in bone metastatic samples that were downregulated or unchanged at metastatic lesions of other sites. Literature analysis of the top differentially expressed genes revealed the functional importance of ITGB3 in its role as the critical subunit of integrin αVβ₃ in both prostate cancer cell homing to bone and in homotypic binding between cells. ITGB3 mRNA expression level was compared in the top three metastatic sites and bone had the most significantly increased expression level compared to lymph node and liver. cBioPortal was used to access the Metastatic Prostate Cancer Patient database and SU2C/PCF Dream Team Cancer study was used as the primary database. The genomic profiles that were selected were mRNA expression data/capture z-Scores (RNA Seq capture).

Cell Culture. C4-2B cells were purchased from MD Anderson Characterized Cell Line Core Facility (Houston, Tex.) and LNCaP cells were purchased from ATCC (Manassas, Va.). Both cells lines were maintained in standard cell culture conditions (5% CO₂, 37° C.) and cultured in RPMI-1640 medium, 10% fetal bovine serum, and 1% antibiotic-antimycotic (Gibco).

αVβ₃ Protein Stimulation and Verification. C4-2B cells were grown on glass coverslips in a six-well dish and then stimulated to express αVβ₃ by treatment with 200 ng/mL of recombinant human CXCL12 (R&D Systems, Minneapolis, Minn.) for 1, 6, or 12 hours at 37° C. After cells were stimulated they were rinsed with PBS, fixed with 4% formaldehyde (Affymetrix) for 10 min at room temperature, washed twice with PBS, and blocked for 1 hour with 1% BSA in PBS. Next cells were incubated for 4 hours with a 1:100 dilution of anti-integrin αVβ₃ antibody, clone LM609 (Millipore). Incubation was followed by three washes with PBS, and then incubated for 45 minutes with a 1:200 dilution of the goat anti-mouse IgG (H+L) secondary antibody, Alexa Fluor 488 conjugate (Life Technologies). Cells were washed twice more and mounted on slides with Prolong Gold antifade reagent with DAPI (Invitrogen). Fluorescent images were taken with Olympus AX70 Florescent Microscope.

Cancer Coated Nanoparticle Preparation. C4-2B cells were grown to 90% confluency in a T-175 flask and then if P-BiNPs were to be made, they were stimulated with 200 ng/ml recombinant human CXCL12 for 1 hour at 37° C. After stimulation step for P-BiNP or no stimulation for BiNP, cells were prepared similarly to publication by Fang et al. with modifications [10]. Cells were washed with PBS and lifted from flask using 2 mM ethylenediaminetetraacetic acid (EDTA) in PBS. Cells were washed three more times with PBS by centrifugation at 500 g. On final wash, cells were suspended in hypotonic buffer solution consisting of 10 mM KCl, 2 mM MgCl₂, 20 mM Tris-HCl adjusted to pH 7.5. Immediately before hypotonic buffer use, 1 Pierce EDTA-free protease inhibitor tablet (Thermo Scientific) and phosphate inhibitor cocktail (EMD Millipore, USA) were added to 50 mL of the hypotonic buffer. Next, cancer cells in the hypotonic buffer were placed in Dounce homogenizer and mashed 25 times. Homogenized cells were centrifuged at 3200 g for 5 minutes at 4° C. on desktop centrifuge and supernatant was removed and saved on ice. Pellet was suspended in hypotonic buffer and again the Dounce homogenizer was used for 25 mashes. The second homogenate was placed in centrifuge at 3200 g for 5 minutes at 4° C. and supernatant was removed and placed on ice. Pooled supernatant was spun at 20,000 g for 20 minutes. The supernatant was removed and transferred to clean ultracentrifuge tubes and spun at 100,000 g for 16 hours. The supernatant was removed and discarded. Pellet consisting of purified cell membrane fraction was washed in Tris Buffer (10 mM Tris-HCl with 1 mM EDTA adjusted to pH 7.5). The total protein in the membrane fraction was quantified using a Pierce BCA Protein Assay kit (Life Technologies) per manufacturer's instructions. This membrane was then used in further experiments.

PLGA nanoparticles were made using a nanoprecipitation method. Briefly, a 26 G flat tipped needle attached to a 1 mL syringe was used to inject 7.5 mg/mL PLGA 5050 dissolved in acetone (Lakeshore Biopharmaceutics) into sterile water. The acetone was evaporated under nitrogen gas flow for 20 minutes. Nanoparticles were washed 3 times with sterile water in Millipore tubes by centrifuging at 1500×g for 20 minutes. If Cabazitaxel (MedChem Express), Nile Red fluorescent dye (Invitrogen), or IR-780 dye (Sigma Aldrich) was to be used in the experiment, then it was dissolved in the initial PLGA/acetone mixture prior to nanoprecipitation. These nanoparticles were then used as the stock for the core of the cancer coated nanoparticles as described in the procedure below. This ensures that all nanoparticle groups tested had equivalent dye concentrations for a particular experiment.

Nanoparticle coating was performed by an extrusion process. First, the membrane fraction from the cancer cells was adjusted to 1 mg/ml and extruded through a Nuclepore Track-Etch Membrane (Whatman) with 400 nm pore size 11 times using an Avanti Lipids extruder. Nanoparticles were added so that the ratio of membrane protein to nanoparticles (0.5:1) (w/w). This mixture was extruded through a 200 nm membrane 11 more times. Coated nanoparticles may then be washed by centrifugation at 14,000 rpm for 30 min.

Cancer Cell Membrane Fraction Verification. Western Blot was used to verify membrane fraction preparation by comparing protein expression in the pure membrane fraction versus whole cell lysate in two different cell lines. For the whole cell lysate, total protein was extracted from cancer cells and quantified. The membrane fraction used was from the protocol described above. Protein was separated on 4-12% Bis-Tris Nu-PAGE gel (Invitrogen, CA) with MES running buffer. The primary antibodies were against Na⁺/K⁺ ATPase as membrane marker (mouse monoclonal antibody from Developmental Studies Hybridoma Bank, IA), lamin was used as a nuclear marker (Santa Cruz Biotechnology, CA), and cytochrome c was used as a mitochondria marker (Santa Cruz Biotechnology, CA). Appropriate secondary antibodies, diluted to 1:200, and conjugated with horseradish peroxidase (Promega, Wis.) were incubated with membranes for 2 hours at room temperature. Membranes were developed using ECL plus (Amersham Pharmacia Biotech, IL) and images were taken with α-imager Fluortech HD2 (San Jose, Calif.).

Coomassie stain was used to verify that the membrane fraction still maintained a broad profile of protein expressed on the surface of the cells. All initial steps are the same as described in the Western Blot procedure above however after the protein was separated on 4-12% Bis-Tris NuPAGE gel, it was stained with Coomassie Brilliant Blue (BioRad, CA) for 30 minutes. The gel was then destained for 2 days by washing with destain solution (20% methanol, 10% glacial acetic acid, in ddH₂O). The gel was then imaged with the α-imager Fluortech HD2.

Membrane Coating Stability. Various ratios of membrane to nanoparticles were used to determine the optimal ratio needed for complete coating of cancer coated nanoparticles. Nanoparticles were then incubated in PBS solution which will cause non-coated nanoparticles to aggregate. The size was checked after 12 hours by DLS utilizing the Zetasizer Nano ZS instrument (Malvern Ltd). In addition, the inventors assessed whether there was a difference in stability between the P-BiNPs and BiNPs through the same aggregation assay as described above with size and PDI being measured once a day over seven days.

Hydrodynamic Size and PDI. Particle size and PDI of membrane coated and non-coated nanoparticles were measured by dynamic light scattering (DLS) utilizing the Zetasizer Nano ZS instrument (Malvern Ltd).

Zeta Potential. The zeta (ζ) potential of the membrane coated and non-coated nanoparticles were measured using the Zetasizer Nano ZS Instrument. Nanoparticles were loaded into a folded capillary cell (Malvern Ltd.) and zeta potential was determined based on the electrophoretic mobility of the nanoparticles.

Transmission Electron Microscopy. Nanoparticles were prepared for transmission electron microscopy (TEM) by placing a formvar-carbon coated grid in a Pelco easiGlow discharge machine. One drop of the nanoparticles was placed on the grid and left for 1 minute. Liquid was wicked off grid with filter paper. For negative staining, one drop of 1% uranyl acetate was added to the grid and left for 1 minute and then the liquid was wicked off with filter paper. Sample was imaged with the FEI Tecnai G2 Spirit Biotwin Transmission Electron Microscope.

Nanoparticle Uptake in C4-2B Cells. Flow cytometry was used to determine uptake of P-BiNP. PLGA nanoparticles were tagged with Nile red fluorescent dye and coated with membranes. C4-2B cells or human fibroblast cells (HFF1 purchased from ATCC) were plated on six-well dishes at a density of 0.5×10⁶ cells per well and allowed to attach for 24 hours prior to being treated with either: BareNPs, BiNPs, or P-BiNPs for an hour. After incubation, cells were rewashed with PBS and then detached with trypsin. Cells were rewashed at 200×g for 10 min with 1% FBS diluted in PBS. Cells were fixed with 2% PFA for 15 minutes at 4° C. Then 1% FBS in PBS was added to cells followed by centrifugation at 200×g for 10 min. Cells were suspended in 1% FBS in PBS. Beckman Coulter Cytomics FC500 Flow Cytometry Analyzer was gated on red fluorescence channel to determine nanoparticle uptake.

Simultaneous uptake of cell membrane coating and nanoparticle core was additionally studied. C4-2B cells were plated on glass coverslips in six-well plates at density described above and allowed to attach for 24 hours. Nanoparticles were prepared by incorporating Nile red into the core PLGA as described above. Cancer cell membrane fraction was tagged with Pkh26 (Sigma Aldrich) per manufacturer's protocol prior to the first extrusion. Nanoparticles were added to cell culture media for 1 hour. Excess nanoparticles were washed out thrice with PBS. Cells were fixed with 4% paraformaldehyde for 10 minutes, washed with PBS, then mounted on microscope slides with Prolong Gold anti-fade reagent with DAPI (Invitrogen). Cells were imaged with the Zeiss LSM 510 confocal microscope.

For spheroids generation, C4-2B and HFF-1 cell lines were combined at a 3:1 ratio and plated at 3×10⁶ cell/mL in Aggrewell 800 plates (Stem Cell Technologies) following manufacturer's instructions. Aggregates were allowed to form over 24 hrs in Aggrewells followed by 24 hrs in ultralow six-well plates on an orbital shaker. Resulting spheroids were incubated with BareNP or P-BiNP loaded with Nile Red dye for 3 hrs in a 1.5 mL microcentrifuge tube at 37° C. and 5% CO₂ with gentle shaking. Nanoparticles were removed from solution by washing spheroids and fixed in 4% paraformaldehyde for 30 min. Samples were washed in PBS, then in 100 μL of 100% methanol incubation for 15 min. Samples were further processed by adding 20% DMSO in 100% methanol for 2 minutes and repeated, then 80% methanol in PBS for 2 minutes, 50% methanol in PBS for 2 minutes, PBS alone for 2 minutes twice, and finally twice in 1% TritonX™ 100 in PBS for 2 minutes. Samples were then placed in penetration buffer consisting of 0.2% Triton/0.3 M glycine/20% DMSO in PBS for 15 minutes and blocked with 0.2% TritonX/6% donkey serum/10% DMSO in PBS at 37° C. with gentle shaking for 15 minutes. Spheroids were finally stained with DAPI and imaged on Keyence BZ-X700.

Cell Viability Assay. C4-2B cells were plated on 96 well flat bottom plates (Corning Incorporated Durham, N.C.) at a density of 2000 cells per well. Cells were allowed to attach for 24 hours then treated with increasing concentrations (0-20 μg/ml Cabazitaxel loaded) BiNP or P-BiNP for 72 hours in standard cell culture conditions. At respective time points 20 μl Thiazolyl Blue Tetrazolium Bromide (MTT) (Sigma, St. Louis, Mo.) suspended in PBS at a concentration of 5 mg/mL was added to the 96 well plate. After three hours of incubation, media was removed and 100 μl of DMSO was added to all wells and mixed by pipetting. Absorbance was read on BioTek Synergy 2 Multi-Mode Plate Reader (Winooski, Vt.) at 570 nm. Percentage cell viability was calculated by dividing absorbance of sample by the average of untreated cells in quadruplicate and then multiplied by one hundred.

In vivo bone homing. Male athymic nude-foxn1nu were injected intravenously with 100 μl of freshly prepared saline, dye, BiNP, or P-BiNP via lateral tail vein injection. Treatment groups were prepared as described above with incorporation of IR-780 dye (Sigma-Aldrich, USA) into the core of the nanoparticle (similar to described above encapsulation of Nile red into nanoparticle core) prior to coating or the equivalent concentration of dye used in dye only group to ensure consistency among groups. Two hours after injection mice were sacrificed and organs excised and imaged with IVIS animal imager: excitation 725, emission 775 (Perkins Elmer, USA). For higher resolution scans and more sensitive detection of NIR signal in bone, lower limbs were imaged separately and fluorescent signal quantified with Odyssey CLx (LI-COR, USA).

In vivo bone retention. Male athymic Nude-Foxn1nu were injected intraosseously into the tibia with 10 μl saline, dye, BiNP, or P-BiNP (all with IR-780 encapsulated within the core of the nanoparticle) with a 28G needle. Animals were imaged initially and at time points: 1 hour, 24 hours, 48 hours, and 72 hours to assess for NIR signal on IVIS animal imager. Fluorescent signal was quantified at each time and compared to the signal for the initial time point of each mouse to give the percentage of retained nanoparticle in the bone. Additionally, half-life of nanoparticle signal in the bone was calculated with the following equation: N(t)=N₀(½)\hat{ }(t/t_{1/2}).

Cytotoxicity of PBiNPs on normal cells. To examine the cytotoxic effect of the PBiNPs against normal prostate epithelial cells (PWR1E), cell toxicity assay was performed. Normal epithelial prostate cells were treated with highest dose of the PBiNPs for 24 hours.

Western blot Analysis for Expression of Proteins. Cells were lysed in NP-40 buffer containing protease and phosphatase inhibitors cocktail (EMD Millipore, Billerica, Mass.). Protein concentrations were determined by Pierce BCA protein assay kit (Thermo Scientific, Rockford, Ill.). Cell extracts were separated on 4-12% Bis-Tris NuPAGE gel (Life Technologies Corporation, Carlsbad, Calif.) using MES buffer and transferred onto nitrocellulose membrane. Membranes were blocked with 5% fat-free milk in Tris-buffered saline containing 0.05% Tween 20 (TBST) at room temperature for 60 min, and incubated overnight at 4° C. with the appropriate primary antibody in 5% milk in TBST. After three washings with TBST, the membrane was incubated with the appropriate secondary antibody (Southern Biotech, Birmingham, Ala.) at room temperature for 2 h. After washing again with TBST, the membranes were developed using Immobilon Western Chemiluminescent HRP substrate (Millipore Corporation, Billerica, Mass.), and the image was captured using alpha-imager Fluoretech HD2.

### REFERENCES

1. Danhier et al., J Control Release 2010, 148 (2), 135-46.

2. Bertrand et al., Adv Drug Deliv Rev 2014, 66, 2-25.

3. Peer et al., Nature nanotechnology 2007, 2 (12), 751-60.

4. Elias et al., Nanomedicine: nanotechnology, biology, and medicine 2013, 9 (2), 194-201.

5. Marusyk et al., Nature reviews. Cancer 2012, 12 (5), 323-34.

6. Gdowski et al., J Exp Clin Cancer Res 2017, 36 (1), 108.

7. Hu et al., PNAS 2011, 108 (27), 10980-5.

8. Jin et al., International journal of cancer 2011, 128 (11), 2545-61.

9. Sun et al., The Prostate 2007, 67 (1), 61-73.

10. Fang et al., Nano letters 2014, 14 (4), 2181-8.

11. Glinskym et al., Cancer research 2003, 63 (13), 3805-11.

12. Khaldoyanidi et al., The Journal of biological chemistry 2003, 278 (6), 4127-34.

13. Li et al., ACS nano 2017, 11 (7), 7006-7018.

14. Sarveswaran et al., PloS one 2015, 10 (4), e0122805.

15. Thalmann et al., Cancer research 1994, 54 (10), 2577-81.

16. Luk et al., Nanoscale 2014, 6 (5), 2730-7.

17. Hines and Kaplan, Critical reviews in therapeutic drug carrier systems 2013, 30(3), 257-276.

18. Zhu et al., Nano letters 2016, 16(9), 5895-901.

19. de Leeuw et al., Clinical cancer research 2015, 21(4), 795-807.

20. Wong et al., Clinical & experimental metastasis 1998, 16 (1), 50-61.

21. van der Pluijm et al., Laboratory investigation 1997, 77 (6), 665-75.

22. Sung et al., Journal of cellular physiology 1998, 176 (3), 482-94.

23. Noti, International journal of oncology 2000, 17 (6), 1237-43.

24. Kodeck et al. Cell Reports 2012, 21, 3298-309.

25. Lee et al. Oncotarget 2015, 6(28), 25619-30.

26. Gdowski et al. Journal: ACS Applied Nano Materials, 2019.

1. A programmed delivery vehicle comprising a programmed membrane encapsulating a cargo.

2. The programmed delivery vehicle of claim 1, wherein the programmed membrane comprises 1, 2, 3, 4, 5, 6, 7, 8, 9, 10 or more target cell surface proteins.

3. The programmed delivery vehicle of claim 2, wherein at least one of the target cell surface proteins is an integrin.

4. The programmed delivery vehicle of claim 1, wherein the programmed membrane is derived from a source cell treated or transfected with an expression construct producing a programmed cell surface profile on the source cell.

5. The programmed delivery vehicle of claim 4, wherein the cell surface profile is a metastatic cancer cell surface profile.

6. The programmed delivery vehicle of claim 5, wherein the metastatic cancer cell surface profile is a bone metastasis profile, a liver metastasis profile, a brain metastasis profile, or a lymph node metastasis profile.

7. The programmed delivery vehicle of claim 1, wherein the cargo is a nanoparticle, a chemotherapy, a drug, an imaging agent, or combination thereof.

8. A method of treating a subject comprising administering to the subject a programmed delivery vehicle of claim 1.

9-15. (canceled)

16. A method for making programmable bioinspired nanoparticles comprising:
(a) identifying gene(s) selectively expressed or overexpressed in a tissue or cell targeted;
(b) obtaining a target cell population and stimulating the target cell population under conditions that increase the expression of one or more identified genes forming a stimulated cell population;
(c) isolating the membranes from the stimulated cell population forming stimulated cell membranes;
(d) coating polymeric nanoparticles with stimulated cell membranes forming a programmed bioinspired nanoparticle (P-BiNP).

17. The method of claim 16 wherein identifying gene(s) selectively expressed or overexpressed includes bioinformatic analysis of RNAseq data from tissues or cells.

18. The method of claim 17, wherein the tissue or cell targeted is a cancer.

19-20. (canceled)

21. The method of claim 16, wherein the gene(s) identified are selectively expressed or overexpressed in a metastasis.

22. The method of claim 21, wherein the metastasis is bone, liver, brain, lymph node, or lung.

23. The method of claim 16, wherein the gene(s) identified include integrin αVβ₃.

24. The method of claim 16, wherein stimulating an isolated target cell population is performed in vitro.

25. The method of claim 16, wherein stimulating an isolated target cell population results in a 2 fold or more increase in expression of a gene identified as being selectively expressed or overexpressed in a tissue or cell targeted.

26. The method of claim 16, wherein stimulating agents, conditions, or agents and conditions increase expression of integrin αVβ₃ in the stimulated cell population.

27. The method of claim 16, wherein the coating of the nanoparticle is by co-extrusion of stimulated cell membranes and nanoparticles.

28. The method of claim 16, wherein P-BiNP is coated at a ratio of 0.25:1 and 1:1 weight of cell membrane protein to weight of nanoparticle.

29. The method of claim 16, wherein the nanoparticles are a cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate, or polycaprolactone nanoparticle.

30-32. (canceled)
